(19) United States
(12) Patent Application Publication
(10) Pub. No.: US 2016/0206230 A1
(43) Pub. Date: Jul. 21, 2016

(54) PERIOD ESTIMATION APPARATUS, PERIOD ESTIMATION METHOD AND STORAGE MEDIUM

(71) Applicant: Oki Electric Industry Co., Ltd., Tokyo (JP)

(72) Inventors: Kohei YAMAMOTO, Tokyo (JP); Motoko KAGAYA, Tokyo (JP); Kurato MAENO, Tokyo (JP)

(73) Assignee: Oki Electric Industry Co., Ltd., Tokyo (JP)

(21) Appl. No.: 14/955,994

(22) Filed: Dec. 1, 2015

(30) Foreign Application Priority Data
Jan. 19, 2015 (JP) 2015-008008

(51) Int. Cl. A61B 5/11; A61B 5/00; A61B 5/113

(52) U.S. Cl. CPC A61B 5/1128; A61B 5/113; A61B 5/7246; A61B 5/7278; A61B 5/7225

(57) ABSTRACT

There is provided a period estimation apparatus including: a first frequency estimation unit that estimates a first frequency of a one-dimensional signal on the basis of a phase difference between the one-dimensional signal and a reference signal and a temporal change in the phase difference; and a second frequency estimation unit that estimates a second frequency of the one-dimensional signal by cutting out a comparative signal having a section length equivalent to a first period corresponding to the first frequency from the one-dimensional signal, and calculating a correlation coefficient between the comparative signal and the one-dimensional signal having the section length.

### CROSS REFERENCE TO RELATED APPLICATION(S)

This application is based upon and claims benefit of priority from Japanese Patent Application No. 2015-008008, filed on Jan. 19, 2015, the entire contents of which are incorporated herein by reference.

### BACKGROUND

The present invention relates to a period estimation apparatus, a period estimation method and a storage medium.

In recent years, technical development on a system for detecting motion of a subject such as breathing and evaluating the motion has been promoted so as to check a health condition of the subject, information on subject's safety, and the like.

For example, according to a technology disclosed in JP 2013-211779A, the number of breaths is measured on the basis of an output signal obtained from a subject by using a Doppler sensor. More specifically, according to the technology disclosed in JP 2013-211779A, Fourier analysis is performed on the Doppler signal that the Doppler sensor has acquired from the subject, a frequency having the highest peak is extracted as a breathing frequency, and the number of breaths is calculated on the basis of the extracted breathing frequency.

According to a technology disclosed in WO 2011/019091, breathing stability is calculated using waveform information based on a breathing waveform so as to achieve a system for evaluating sleep. More specifically, according to the technology disclosed in WO 2011/019091, a waveform of breathing during sleep is extracted, Fourier analysis is performed on the obtained waveform, a frequency of a component having peak amplitude on an amplitude spectrum is detected, and a breathing stability is evaluated on the basis of standard deviation or an average that is an amount of statistics of a temporal change in a value of the frequency.

### SUMMARY

According to JP 2013-211779A and WO 2011/019091, a period of the breathing waveform is calculated on the basis of the frequency spectrum obtained by performing Fourier analysis on waveforms including a plurality of periods. However, there is a problem that it is difficult to accurately calculate a period of a signal for each period, the signal changing infinitesimally for each period such as breathing. More specifically, for example, in a case in which discrete Fourier transform (DFT) is used as Fourier analysis, a section for 10 seconds is necessary to obtain resolution of 0.1 Hz, and unfortunately, a breathing period to be obtained is an average value thereof. Therefore, it is difficult to recognize an infinitesimal fluctuation for each period of breathing.

Accordingly, in a nod to the above described problem, the present invention proposes a novel and improved period estimation apparatus, period estimation method and storage medium capable of calculating a period of a signal with a higher accuracy for each period.

According to an embodiment of the present invention, there is provided a period estimation apparatus including: a first frequency estimation unit that estimates a first frequency of a one-dimensional signal on the basis of a phase difference between the one-dimensional signal and a reference signal and a temporal change in the phase difference; and a second frequency estimation unit that estimates a second frequency of the one-dimensional signal by cutting out a comparative signal having a section length equivalent to a first period corresponding to the first frequency from the one-dimensional signal, and calculating a correlation coefficient between the comparative signal and the one-dimensional signal having the section length.

The first frequency estimation unit may generate the reference signal having a frequency depending on the estimated first frequency

In a case in which the estimated first frequency is not present in a predetermined frequency band, the first frequency estimation unit may estimate, as the first frequency of the one-dimensional signal, a frequency of the reference signal used by the first frequency estimation unit.

The second frequency estimation unit may estimate a second frequency of the one-dimensional signal on the basis of a time lag at a time when the correlation coefficient becomes maximum in a range determined by the first period.

The period estimation apparatus may further includes: a determination unit that determines any of the first frequency and the second frequency as an estimation frequency of the one-dimensional signal on the basis of the first frequency, the second frequency, and the correlation coefficient at a time when the second frequency has been estimated.

The period estimation apparatus may further includes: a Doppler sensor that radiates a radiation wave toward an object, and outputs a beat signal having a frequency of a difference between a frequency of the radiation wave and a frequency of a reflection wave reflected by the object that has received the radiation wave; a beat signal acquisition unit that acquires the beat signal output from the Doppler sensor; and a signal conversion unit that converts the acquired beat signal into the one-dimensional signal.

The period estimation apparatus may further includes: a filter unit that reduces a low-frequency component in the beat signal and outputs a corrected signal obtained by reducing the low-frequency component.

The signal conversion unit may perform principal component analysis on the corrected signal represented as a two-dimensional vector, and converts the corrected signal into the one-dimensional signal having an output value that is a value of a principal component direction of the corrected signal converted by an eigenvector obtained from the principal component analysis.

The signal conversion unit may convert the corrected signal into the one-dimensional signal having an output value that is a solution of a continuous function of a product of strength of the corrected signal and temporal differentiation of an argument of the corrected signal on a two-dimensional surface.

Motion of the object may be breathing motion of a living body.

The period estimation apparatus may further includes: a peak position estimation unit that cuts out a criterion signal having a section length equivalent to a period corresponding to the estimation frequency from the one-dimensional signal, and estimates a peak position of the one-dimensional signal on the basis of a phase difference between the criterion signal and a cosine wave having the estimation frequency and a predetermined initial phase.

The period estimation apparatus may further includes: a peak position deciding unit that extracts respective peak positions estimated in the past in a section of the criterion signal used at a time when the peak position estimation unit has estimated the peak position, and decides the peak position on the basis of distribution of the respective peak positions that have been extracted.

The period estimation apparatus may further includes: a peak interval calculation unit that calculates, as an peak interval, a difference between the two decided peak positions that are consecutive to each other.

According to another embodiment of the present invention, there is provided a period estimation method including: estimating a first frequency of a one-dimensional signal on the basis of a phase difference between the one-dimensional signal and a reference signal and a temporal change in the phase difference; and estimating a second frequency of the one-dimensional signal by cutting out a comparative signal having a section length equivalent to a first period corresponding to the first frequency from the one-dimensional signal, and calculating a correlation coefficient between the comparative signal and the one-dimensional signal having the section length.

According to another embodiment of the present invention, there is provided a storage medium having a program stored therein, the program causing a computer to function as: a first frequency estimation unit that estimates a first frequency of a one-dimensional signal on the basis of a phase difference between the one-dimensional signal and a reference signal and a temporal change in the phase difference; and a second frequency estimation unit that estimates a second frequency of the one-dimensional signal by cutting out a comparative signal having a section length equivalent to a first period corresponding to the first frequency from the one-dimensional signal, and calculating a correlation coefficient between the comparative signal and the one-dimensional signal having the section length.

As described above, according to one or more embodiments of the present invention, it is possible to calculate a period of a signal with a higher accuracy for each period.

### BRIEF DESCRIPTION OF THE DRAWINGS

FIG. 1 is a diagram showing an overview of a period estimation apparatus according to an embodiment of the present invention;

FIG. 2 is a block diagram showing a configuration of the period estimation apparatus according to the embodiment;

FIG. 3 is a block diagram showing a configuration of a frequency estimation unit according to the embodiment;

FIG. 4 is a flow diagram showing an operation example of the period estimation apparatus according to the embodiment;

FIG. 5 is a diagram in which a beat signal received by the period estimation apparatus according to the embodiment is shown on a two-dimensional surface;

FIG. 6 is a diagram showing an example of a locus of a corrected signal obtained after a filter process according to the embodiment;

FIG. 7 is a diagram showing an example of a locus of a corrected signal obtained after a filter process according to the embodiment;

FIG. 8 is a diagram showing an example of a frequency estimation method performed by a second frequency estimation unit according to the embodiment;

FIG. 9 is a flowchart showing an operation example of a determination unit according to the embodiment;

FIG. 10 is a diagram showing an example of a peak position deciding method performed by a peak position deciding unit according to the embodiment;

FIG. 11 is a block diagram showing a hardware configuration of the period estimation apparatus according to the embodiment;

### DETAILED DESCRIPTION OF THE EMBODIMENT(S)

Hereinafter, referring to the appended drawings, preferred embodiments of the present invention will be described in detail. It should be noted that, in this specification and the appended drawings, structural elements that have substantially the same function and structure are denoted with the same reference numerals, and repeated explanation thereof is omitted.

### <1. Overview>

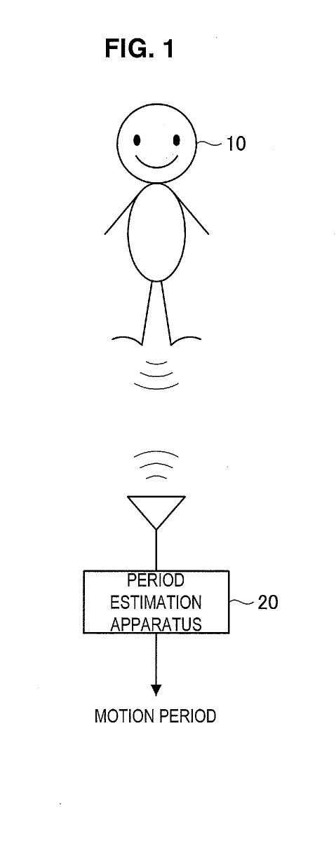

First, with reference to FIG. 1, an overview of a period estimation apparatus according to an embodiment of the present invention is described. FIG. 1 is an explanatory diagram illustrating an overview of a period estimation apparatus according to an embodiment of the present invention.

As shown in FIG. 1, a period estimation apparatus 20 according to the present embodiment radiates a radiation wave such as light, an electromagnetic wave, or an acoustic wave toward a subject 10, and acquires a reflection wave reflected by the subject 10 that has received the radiation wave. The period estimation apparatus 20 includes a Doppler sensor. The Doppler sensor outputs a beat signal (Doppler signal) having a frequency of a difference between a frequency of the radiation wave and a frequency of the reflection wave. The period estimation apparatus 20 estimates a period of the output beat signal.

The period estimation apparatus 20 uses a radiation wave such as light, an electromagnetic wave, or an acoustic wave. Accordingly, the period estimation apparatus 20 can estimate a period of body motion such as breathing without direct contact with the subject 10. Therefore, for example, the period estimation apparatus 20 can detect change in biological information such as breathing of the subject 10 within the reach of the radiation wave.

The Doppler sensor is a sensor for detecting motion of a target object (here, the subject 10) by considering a shift in frequency (Doppler effect) of a reflection wave in proportion to a velocity of the moving target object. Since the beat signal output from the Doppler sensor has a frequency shifted by the Doppler effect, it is possible to detect motion information of the target object by analyzing the beat signal. However, the output beat signal includes not only breathing that is a detection target but also body sway, heartbeat, ambient noise, and the like, for example. Accordingly, it is necessary to detect only waveform information of breathing from the beat signal when the breathing is set as the detection target, for example.

For example, according to the invention disclosed in JP 2013-211779A, Fourier analysis is performed on the output beat signal by using a fast Fourier transform, a frequency having the highest peak in a frequency band equivalent to breathing is set as a frequency of the breathing, and the number of breaths is detected on the basis of the frequency. However, in a case in which the breathing is detected using Fourier analysis, a window size necessary for the Fourier transform is a length of at least a few breaths. Therefore, information of one breath is not detected. In addition, since it is difficult to specify a peak position of a waveform for each breath by Fourier analysis, it is difficult to detect a change in period that changes for each breath.

In view of the foregoing situation, the period estimation apparatus 20 has been conceived. It is possible for the period estimation apparatus 20 according to an embodiment of the present invention to calculate a period of a signal waveform such as breathing with a higher accuracy for each period. Next, details of a configuration of the period estimation apparatus 20 according to the present embodiment are described.

### <2. Configuration>

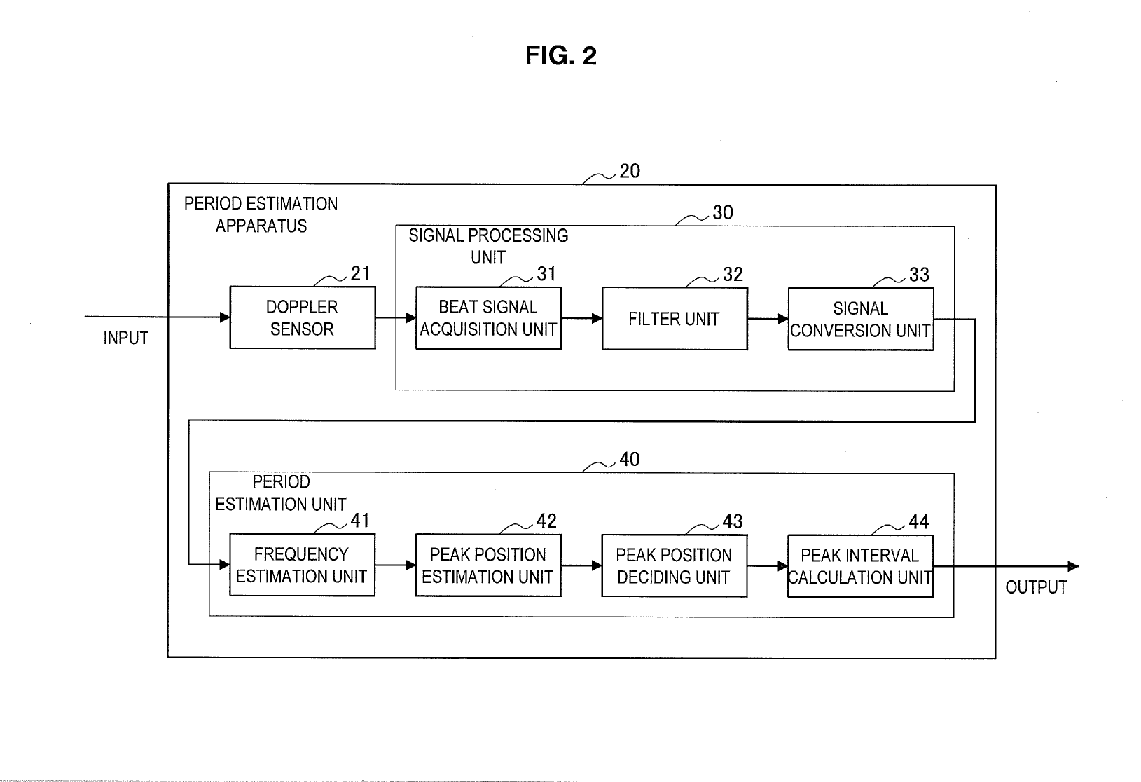

FIG. 2 is a block diagram showing the configuration of the period estimation apparatus 20 according to the present embodiment. As shown in FIG. 2, the period estimation apparatus 20 mainly includes a Doppler sensor 21, a signal processing unit 30, and a period estimation unit 40. The signal processing unit 30 includes a beat signal acquisition unit 31, a filter unit 32, and signal conversion unit 33. The period estimation unit 40 includes a frequency estimation unit 41, a peak position estimation unit 42, a peak position deciding unit 43, and a peak interval calculation unit 44.

The Doppler sensor 21 radiates a radiation wave having a predetermined frequency toward the subject 10, and detects a reflection wave reflected by the subject 10 that has received the radiation wave. According to the Doppler effect, the reflection wave has a frequency shifted from a frequency of the radiation wave depending on motion of the subject 10. The Doppler sensor 21 outputs a beat signal having a frequency of a difference between the frequency of the radiation wave and the frequency of the reflection wave by integrating the reflection wave and the radiation wave by an internal mixer and performing low-pass filtering on the integrated waves. The Doppler sensor 21 may incorporate quadrature detection. In this case, the Doppler sensor 21 outputs two kinds of beat signals including a cosine wave component (I component) and a sine wave component (Q component). Note that, “I” as used herein means in-phase, and “Q” as used herein means quadrature.

The beat signal acquisition unit 31 has a function of acquiring the beat signal output from the Doppler sensor 21. The acquired beat signal is output to the filter unit 32.

The filter unit 32 has a filter function of reducing a low-frequency component such as a direct-current component in the beat signal output from the beat signal acquisition unit 31. Subsequently, the filter unit 32 outputs, as a corrected signal, the beat signal whose low-frequency component has been reduced by the filter function to the signal conversion unit 33. Note that, the filter unit 32 can use various filters such as a high-pass filter, a band-pass filter, and an IIR filter, or may use a combination thereof.

The signal conversion unit 33 has a function of converting the corrected signal output from the filter unit 32 into a one-dimensional signal. The one-dimensional signal acquired by converting the corrected signal is output to the frequency estimation unit 41. Details of a specific function of the signal conversion unit 33 are described in the operation example. The one-dimensional signal as used herein is a time-series signal, for example. More specifically, the one-dimensional signal is a single signal that changes over time. The single signal includes a real number, an imaginary number, a complex number, and the like. In the following description, a plurality of one-dimensional signals may be referred to as a one-dimensional signal.

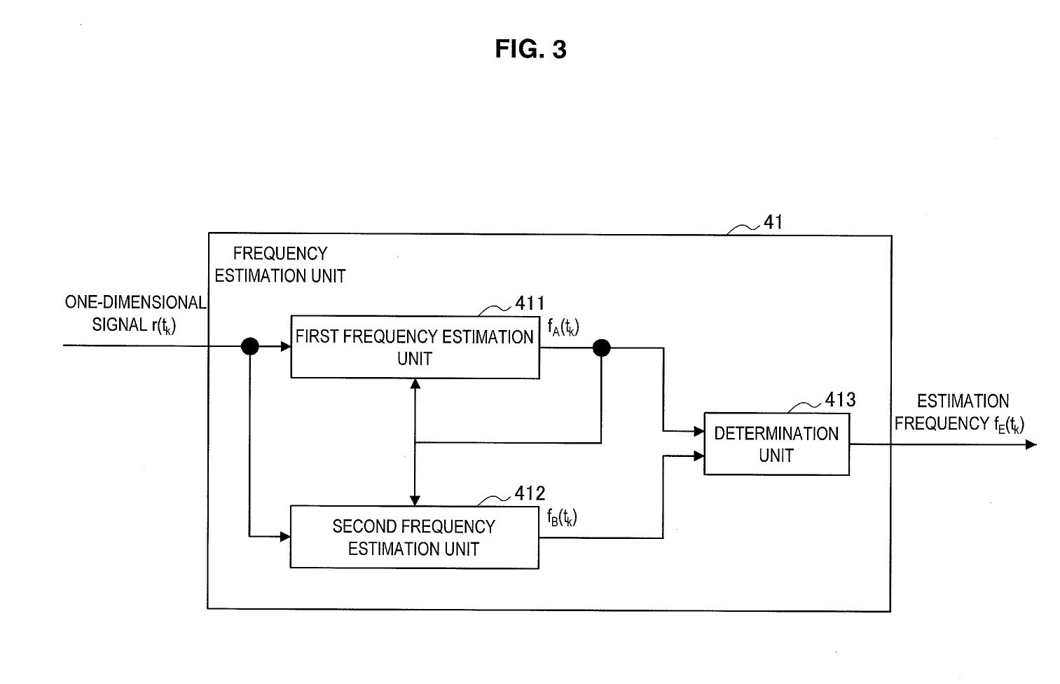

The frequency estimation unit 41 has a function of estimating a frequency of the one-dimensional signal. FIG. 3 is a diagram illustrating a block diagram showing a configuration of the frequency estimation unit 41. The frequency estimation unit 41 includes a first frequency estimation unit 411, a second frequency estimation unit 412 and a determination unit 413. First, the one-dimensional signal is input to the first frequency estimation unit 411 and the second frequency estimation unit 412. Subsequently, the determination unit 413 determines any of a first frequency estimated by the first frequency estimation unit 411 and a second frequency estimated by the second frequency estimation unit 412 as an estimation frequency of the one-dimensional signal, and outputs any of the first frequency and the second frequency to the peak position estimation unit 42. The first frequency estimated by the first frequency estimation unit 411 is fed back to the first frequency estimation unit 411 and the second frequency estimation unit 412. Details of a specific function are described in the operation example.

The peak position estimation unit 42 has a function of estimating a peak position of the one-dimensional signal by using the estimation frequency output from the frequency estimation unit 41. Details of a specific function are described in the operation example.

With respect to the peak position estimated by the peak position estimation unit 42, the peak position deciding unit 43 has a function of deciding the peak position of the one-dimensional signal by using distribution of peak positions that have been estimated in the past. Details of a specific function are described in the operation example.

The peak interval calculation unit 44 has a function of calculating a difference between the two decided peak positions that are consecutive to each other and that have been acquired in the peak position deciding unit 43. The difference between the peak positions acquired in the peak interval calculation unit 44 is output as a period of motion of the object.

The period estimation apparatus 20 may include a storage unit (not illustrated) that stores information such as a signal, frequency, and the like that have been acquired by processes performed by the above described functional parts. Specifically, the storage unit stores information such as a signal, frequency, and the like that have been acquired by the functional parts. The functional parts call up information necessary for fulfill their functions from the storage unit. For example, in the following operation example, the signal conversion unit 33 and the peak position deciding unit 43 can convert a beat signal into a corrected signal and decide a peak position of a one-dimensional signal by using information on past signals and peak positions stored in the storage unit.

The configuration of the period estimation apparatus 20 according to the present embodiment has been described. According to the present embodiment, the period estimation apparatus 20 converts a beat signal acquired from the Doppler sensor into a one-dimensional signal, estimates a frequency of the one-dimensional signal by using two kinds of means, decides a peak position of the one-dimensional signal, and calculates a period for each period. In this way, it is possible for the period estimation apparatus 20 to accurately detect a period of motion such as breathing for each period.

### <3. Operation Example>

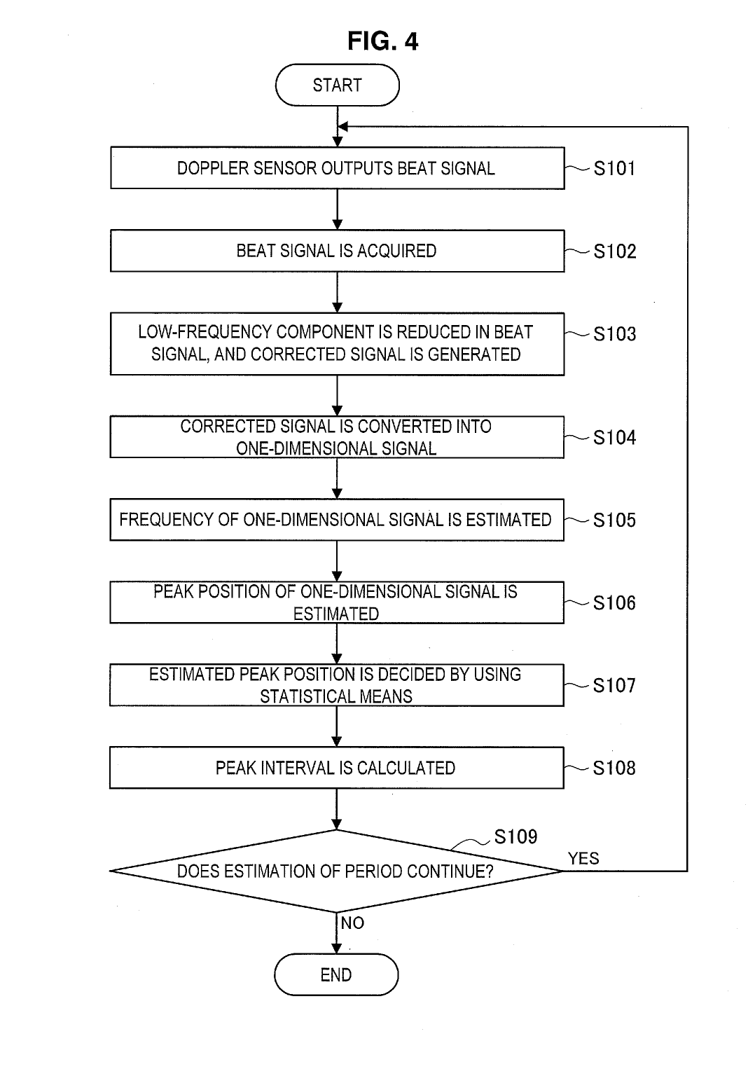

Next, with reference to FIGS. 4 to 10, the operation example of the period estimation apparatus 20 according to the present embodiment is described. FIG. 4 is a flow diagram showing an operation example of the period estimation apparatus 20 according to the present embodiment. Hereinafter, the operation example of the period estimation apparatus 20 according to the present embodiment is described in three steps including “Acquisition and Conversion of Beat Signal”, “Estimation of Frequency”, and “Estimation of Peak Position”.

### (Acquisition and Conversion of Beat Signal)

First, the Doppler sensor 21 generates a beat signal D(t) on the basis of a radiation wave and a reflection wave reflected by the subject 10 that has received the radiation wave, and outputs the beat signal D(t) (S101). The Doppler sensor 21 radiates the radiation wave such as light, an electromagnetic wave, or an acoustic wave, and receives the reflection wave reflected by the subject 10 that has received the radiation wave. The Doppler sensor 21 generates the beat signal D(t) having a frequency of a difference between a frequency of the radiation wave and a frequency of the reflection wave by mixing the received reflection wave and radiation wave. The beat signal D(t) includes components of two waves including the I component and the Q component. The beat signal D(t) is expressed by the following Formula 1, when A(t) represents amplitude, λ represents wavelength, d(t) represents a distance between the Doppler sensor 21 and the subject 10 at a time φ₀ represents an initial phase, O represents a direct-current component, and w represents a noise component.

$$D ( t ) = A ( t ) exp [ - j ( \frac{4 π}{λ} d ( t ) + {φ}_{0} ) ] + O + w \qquad Formula 1$$

Next, the beat signal acquisition unit 31 acquires the beat signal D(t) output from the Doppler sensor 21 (S102). The acquired beat signal D(t) is output to the filter unit 32. Note that, the beat signal acquisition unit 31 may stores the acquired beat signals D(t) in the storage unit in a chronological order.

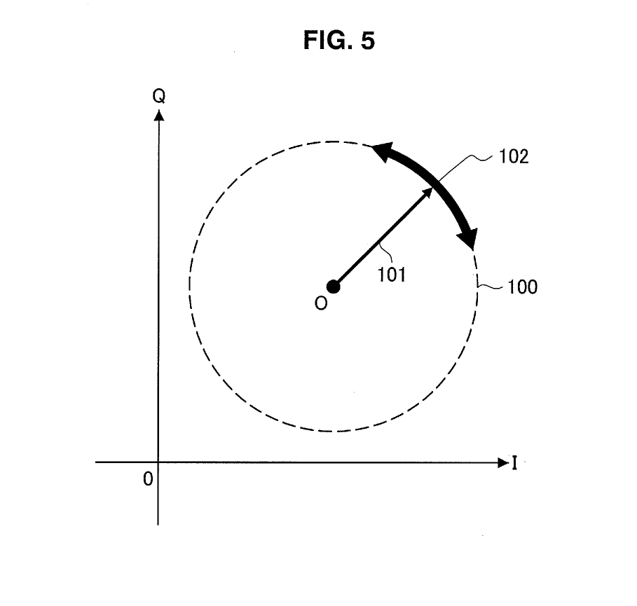

FIG. 5 is a diagram in which the beat signal D(t) is shown on a two-dimensional surface formed by the I component and the Q component. A phase change in the beat signal D(t) is represented by rotation of a circle 100 centered at the direct-current component O. A vector 101 is a vector of the beat signal D(t) whose starting point is the direct-current component O on the two-dimensional surface. A reflection wave reflected by a stationary object such as a wall existing around the subject 10 has a frequency identical to that of the radiation wave, and therefore there is no Doppler effect. Accordingly, the direct-current component O having only a stationary phase difference between the radiation wave and the reflection wave is present as a background of the beat signal D(t), and is set as the starting point of the vector 101 on the two-dimensional surface formed by the I plane and the Q plane. In response to breathing motion of the subject 10, the vector 101 vibrates along an arc 102. The length of the arc 102 is proportional to an amount of change in the distance between the subject 10 and the Doppler sensor 21. In addition, rapidity of change in the vector 101 is proportional to a speed of the breathing motion.

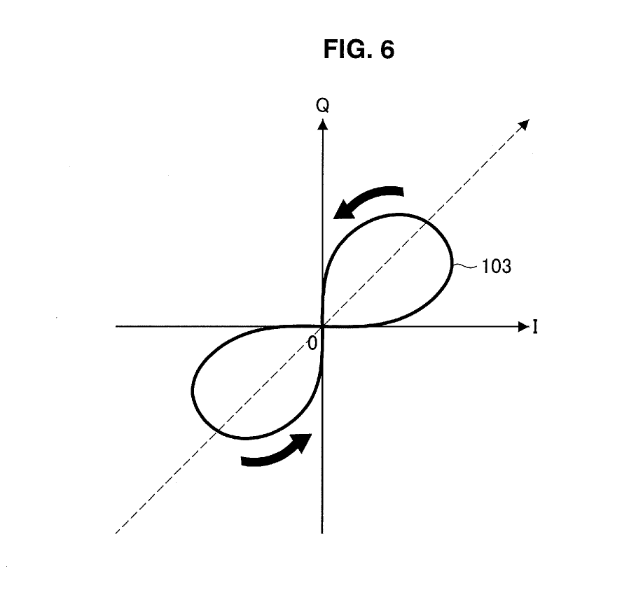

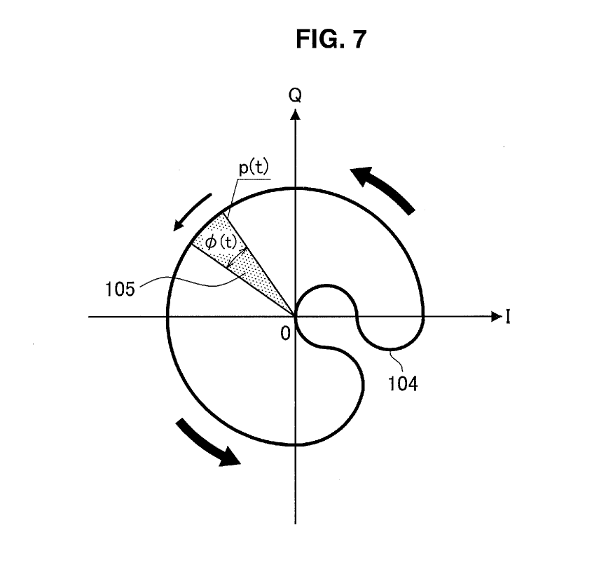

Next, the filter unit 32 reduces a low-frequency component such as the direct-current component O in the beat signal D(t) acquired by the beat signal acquisition unit 31, and outputs a corrected signal in which the low-frequency component has been reduced (S103). By reducing the low-frequency component and the like in the beat signal D(t), the filter unit 32 removes components unrelated to the breathing motion such as the ambient noise and the body motion. For example, a filter used by the filter unit 32 may be various filters such as a high-pass filter, a band-pass filter, and an IIR filter, or a combination thereof. Although details of the corrected signal are described later, the corrected signal draws various loci on the two-dimensional surface formed by the I component and the Q component, as shown in FIGS. 6 and 7, for example.

Next, the signal conversion unit 33 converts the corrected signal output from the filter unit 32 into a one-dimensional signal r(t) (S104). More specifically, the signal conversion unit 33 generates the one-dimensional signal r(t) depending on the output corrected signal by using a first conversion means (to be described later), a second conversion means (to be described later), or a combination of the first conversion means and the second conversion means. By converting the corrected signal serving as a two-dimensional signal into the one-dimensional signal, it is possible to easily conduct subsequent period estimation.

Each of FIGS. 6 and 7 is a diagram in which the locus of corrected signal of the beat signal D(t) output from the filter unit 32 is shown on the two-dimensional surface formed by the I component and the Q component. FIG. 6 shows a locus 103 of the corrected signal output in a case in which the breathing motion is small, in other words, in a case in which the arc 102 shown in FIG. 5 does not travel around the entire circumference of the circle 100. The locus 103 has a figure-of-eight shape through the origin. In a case in which the corrected signal draw the locus 103 as shown in FIG. 6, the signal conversion unit 33 serves as the first conversion means to consider the locus 103 of the corrected signal as a two-dimensional vector from the origin and perform principal component analysis on the corrected signal. More specifically, the signal conversion unit 33 calls up corrected signals for about one period of past breathing existing on the locus 103 from the storage unit, creates a variance-covariance matrix from the two-dimensional vectors of corrected signals, and calculates an eigenvalue and an eigenvector of the matrix. Subsequently, the signal conversion unit 33 outputs an output value of a signal acquired by integrating the corrected signal and the eigenvector, as the one-dimensional signal r(t). In FIG. 6, a principal component direction of the locus 103 is an axial direction of a dashed line. The signal conversion unit 33 continuously outputs values of the principal component direction of the locus 103 to generate the one-dimensional signal r(t). Note that, in the principal component analysis, the principal component direction includes two kinds of components including a first principal component and a second principal component. The signal conversion unit 33 adopts one of the principal components having a larger eigenvalue corresponding to each principal component direction as a principal component direction used for generating the one-dimensional signal r(t).

The principal component analysis method may be an analysis method other than the method for calculating the eigenvalue and the eigenvector of the variance-covariance matrix of the vector of the corrected signal. For example, the principal component analysis method may be a method for calculating principal components dispersing to the maximum in the I component and the Q component of the vector, for example.

On the other hand, FIG. 7 shows a locus 104 of a corrected signal output in a case in which the breathing motion is large. In the case in which the breathing motion is large like deep breathing, the arc 102 drawn by the vector 101 in FIG. 5 sometimes travels around the entire circumference of the circle 100 more than once. In this case, for example, the locus 104 of the corrected signal as shown in FIG. 7 can be obtained. Even if the principal component analysis is performed on the locus 104, it is difficult to detect an accurate principal component. This is because only a low eigenvalue can be obtained in the principal component analysis and the weight of the output value in the principal component direction becomes small in the case in which the corrected signal is distributed around a circle like locus 104. Accordingly, in a case in which it is difficult to obtain an accurate waveform of the one-dimensional signal r(t) by the principal component analysis, the signal conversion unit 33 uses, as the second conversion means, a function of a product of signal strength p(t) and temporal differentiation θ′(t) of an argument θ(t) of the corrected signal on the two-dimensional surface to convert the corrected signal into the one-dimensional signal r(t). Specifically, the signal conversion unit 33 converts the corrected signal by using a function based on a continuous function in the following Formula 2 (sigmoid function in the present embodiment) and setting the one-dimensional signal r(t) as the output value. Note that, in Formula 2, a, K, and c are constant. However, they may be changed as necessary.

r(t)=K[1+exp(aKp(t)θ′(t)−c)]⁻¹ Formula 2

The product of the signal strength p(t) and the temporal differentiation θ′(t) of the argument θ(t) is equivalent to areal velocity of the region 105 shown in FIG. 7. The areal velocity is equivalent to an amount of the breathing motion. The temporal differentiation θ′(t) has a positive and negative value depending on a direction of the breathing motion, and therefore represents a vibration direction of the breathing. Accordingly, the product of p(t) and θ′ (t) accurately represents a state of the breathing motion.

The signal conversion unit 33 converts the corrected signal into a one-dimensional signal r(t) by using the two conversion means. The signal conversion unit 33 may use one of the two conversion means, or may use a combination of the two conversion means. Specifically, in the present embodiment, it is determined which conversion means the signal conversion unit 33 uses on the basis of the amount of the breathing motion, in other words, a length of the arc 102 shown in FIG. 5. For example, in a case in which the breathing motion is small, in other words, in a case in which a length of a locus of the arc 102 is shorter than a circumference of the circle 100, signal strength p(t) and temporal differentiation θ′(t) of an argument θ(t) of the corrected signal becomes small, and therefore the one-dimensional signal r(t) is not accurately represented by the second conversion means. Accordingly, in the case in which the breathing motion is small, the first conversion means is used. On the other hand, in a case in which the breathing motion is large, in other words, in a case in which a locus of the arc 102 shown by the vector 101 vibrates and travels around the entire circumference of the circle 100 more than once, it is difficult to obtain the accurate one-dimensional signal r(t) by using the principal component analysis serving as the first conversion means, and therefore the one-dimensional signal r(t) is obtained by using the second conversion means. It may be determined which of the first conversion means and the second conversion means is used in the signal conversion unit 33, for example, on the basis of the length of the arc 102 representing the locus of the beat signal D(t), or on the basis of an eigenvalue obtained by the first conversion means.

### (Estimation of Frequency)

Next, the operation example at a time of estimating a frequency of the one-dimensional signal r(t) according to the present embodiment is described. In this operation example, the frequency estimation unit 41 estimates the frequency of the one-dimensional signal r(t) (S105). First, the one-dimensional signal r(t) output from the signal conversion unit 33 is input to the frequency estimation unit 41. Specifically, the one-dimensional signal r(t) is input to the first frequency estimation unit 411 and the second frequency estimation unit 412 shown in FIG. 3.

First, the first frequency estimation unit 411 is described. Here, a specific timing when a period is estimated is set to tₖ. The first frequency estimation unit 411 integrates a reference signal and a one-dimensional signal r(tₖ) input at a time of tₖ, extracts a low-frequency component of the integrated signal by using a low-pass filter or the like, and calculates a phase difference φ(tₖ) between the one-dimensional signal r(tₖ) and the reference signal. The reference signal used herein is a signal serving as a model showing a change in a signal caused by one breath. For example, the model may be a model serving as an ideal breathing signal or a model serving as an approximative breathing signal. In the present embodiment, a sine wave or cosine wave having a phase and its temporal change as parameters is used as the reference signal to easily estimate a frequency from a phase change. Accordingly, the reference signal in the present embodiment is a signal of a sine wave or cosine wave having a first estimation frequency f_{A}(tₖ₋₁) estimated by the first frequency estimation unit 411 at a time of tₖ₋₁.

Here, the first frequency estimation unit 411 calculates a change between a phase difference φ(tₖ) and a phase difference φtₖ₋₁) calculated at a time of tₖ₋₁. On an assumption that a temporal change φ(tₖ)−φ(tₖ₋₁) between the phase differences occurs due to a change in the frequency of the one-dimensional signal r(t), the first frequency estimation unit 411 adds a frequency equivalent to the temporal change φ(tₖ)−φ(tₖ₋₁) between the phase differences to the frequency f_{A}(tₖ₋₁) of the reference signal to output a first estimation frequency f_{A}(tₖ).

However, in a case in which the temporal change between the phase differences exceeds a predetermined threshold, an estimated feedback value of a frequency tends to become large, and therefore the first estimation frequency f_{A}(tₖ) unfortunately does not converge with vibration. In view of such problem, the frequency to be added to the frequency f_{A}(tₖ₋₁) of the reference signal may be obtained by integrating a coefficient less than 1 and a frequency equivalent to the temporal change φ(tₖ)−(tₖ₋₁) between the phase differences. By performing such process, f_{A}(tₖ) can converge relatively quickly.

In a case in which the calculated first estimation frequency f_{A}(tₖ) is not present in a predetermined frequency band, the frequency f_{A}(tₖ₋₁) of the reference signal may be output as the first estimation frequency f_{A}(tₖ). For example, in the present embodiment, the predetermined frequency band means a frequency band equivalent to a period of the breathing motion. Specifically, on an assumption that the number of breaths in one minute is 12 to 60, the first estimation frequency f_{A}(tₖ) have to satisfy 0.2 Hz<f_{A}(tₖ)<1 Hz.

The first frequency estimation unit 411 may be achieved by a so-called phase-locked loop (PLL) circuit.

Next, the second frequency estimation unit 412 is described. The second frequency estimation unit 412 performs correlation analysis on the input one-dimensional signal r(t) to estimate a second frequency f_{B}(tₖ) having an accuracy greater than that of the first frequency f_{A}(tₖ) obtained by the first frequency estimation unit 411.

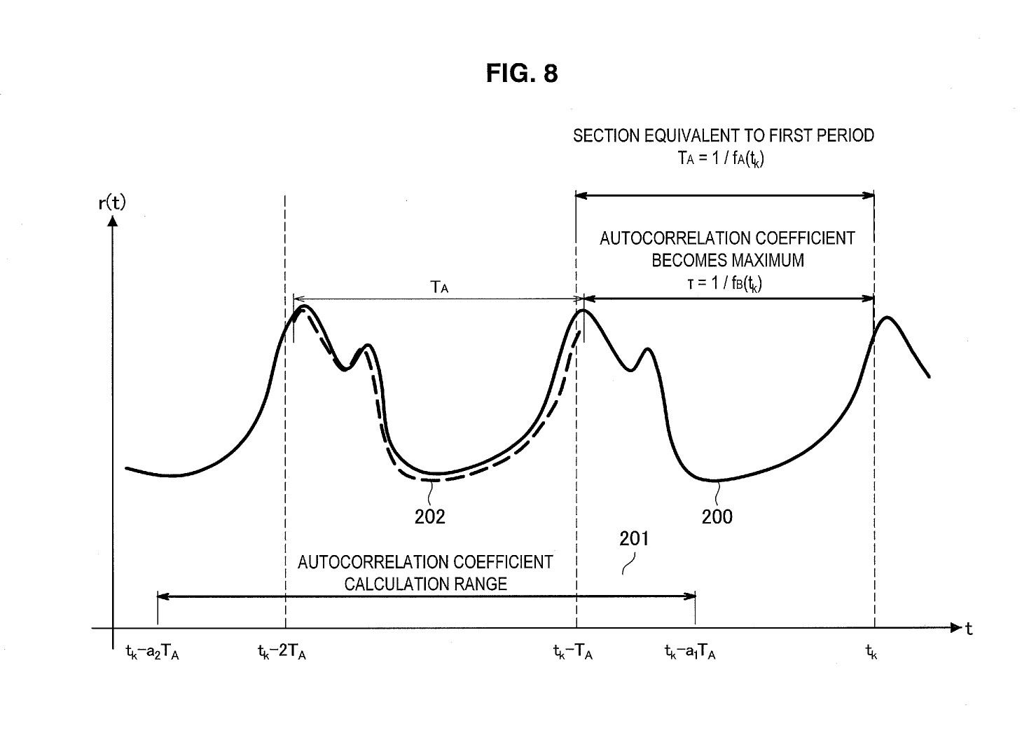

With reference to FIG. 8, a frequency estimation means in the second frequency estimation unit 412 is described. A curve 200 in FIG. 8 is the one-dimensional signal r(t). First, the second frequency estimation unit 412 cuts out a section between the time tₖ-T_{A} and the time tₖ from the one-dimensional signal r(t), and uses it as a comparative signal. T_{A} is a period (first period) corresponding to the first frequency f_{A}(tₖ₋₁) estimated before. Next, the second frequency estimation unit 412 calculates a correlation coefficient using the cut-out comparative signal. As shown in FIG. 8, a correlation coefficient calculation range 201 is equivalent to a range between tₖ-a₂T_{A} and tₖ-a₁T_{A}. Values of a₁ and a₂ may be set arbitrarily. Preferably, the values of a₁ and a₂ include a range between tₖ-2T_{A} and tₖ-T_{A}. The second frequency estimation unit 412 calculates a time lag τₘₐₓ at a time when a correlation coefficient R(τ) between the reference signal 202 and the one-dimensional signal r(t) having a section length T_{A} becomes maximum in the correlation coefficient calculation range 201, and stores the correlation coefficient (τₘₐₓ) at the time of τ=τₘₐₓ in the storage unit. The correlation coefficient R(τ) is calculated using the following Formula 3. T_{c} and T_{d} represents time of a starting point and end point of the comparative signal. With regard to T_{c} and T_{d}, T_{d}−T_{c}=T_{A}.

$$R ( τ ) = \frac{\frac{1}{{T}_{A}} {\int }_{{T}_{c}}^{{T}_{d}} r ( t ) r ( t + τ ) d t}{\sqrt{\frac{1}{{T}_{A}} {\int }_{{T}_{c}}^{{T}_{d}} {r ( t )}^{2} d t} \sqrt{\frac{1}{{T}_{A}} {\int }_{{T}_{c}}^{{T}_{d}} {r ( t + τ )}^{2} d t}} \qquad Formula 3$$

The second frequency estimation unit 412 calculates a second estimation frequency f_{B}(tₖ) from the time lag τₘₐₓ at the time when the correlation coefficient calculated in the correlation coefficient calculation range 201 becomes maximum. f_{B}(tₖ) is calculated using the following Formula 4.

$${f}_{B} ( {t}_{k} ) = \frac{1}{{τ}_{max}} \qquad Formula 4$$

In a range based on a frequency equivalent to the already obtained first estimation frequency f_{A}(tₖ), the second estimation frequency f_{B}(tₖ) obtained above is estimated by correlation analysis on a waveform of the one-dimensional signal r(t). Accordingly, the second frequency estimation unit 412 can estimate a frequency of the one-dimensional signal r(t) more accurately than the first estimation frequency f_{A}(tₖ) obtained from the phase difference from a simple sine wave or cosine wave. However, for example, in a case in which a waveform of breathing drastically changes for each breath, the second estimation frequency f_{B}(tₖ) obtained by the second frequency estimation unit 412 may differ from the first estimation frequency f_{A}(tₖ). In addition, in a case in which R(τₘₐₓ) obtained by the second frequency estimation unit 412 is a low value even if the value of the obtained second estimation frequency f_{B}(tₖ) is close to the first estimation frequency f_{A}(tₖ), the correlation analysis performed by the second frequency estimation unit 412 may be wrong.

Accordingly, the determination unit 413 determines which of f_{A}(tₖ) and f_{B}(tₖ) is adopted as the estimation frequency f_{B}(tₖ) from the first estimation frequency f_{A}(tₖ), the second estimation frequency f_{B}(tₖ), and the correlation coefficient R(τₘₐₓ) at the time when the second estimation frequency has been estimated.

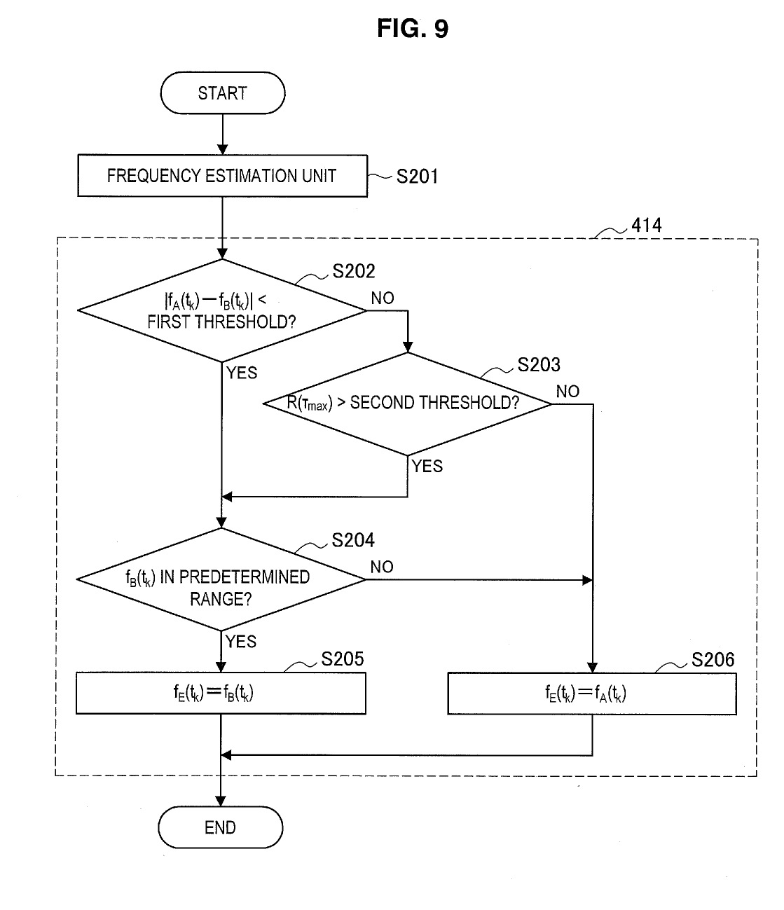

FIG. 9 is a flowchart showing an operation example of the determination unit 413. First, the first estimation frequency f_{A}(tₖ) and the second estimation frequency f_{B}(tₖ) are estimated in the respective frequency estimation units (S201). The estimated frequencies are input to the determination unit 413.

Next, the determination unit 413 determines whether a difference between f_{A}(tₖ) and f_{B}(tₖ) is less than a first threshold (S202). In a case in which the difference between f_{A}(tₖ) and f_{B}(tₖ)is not less than the first threshold (NO in S202), the determination unit 413 calls up R(τₘₐₓ) at the time of tₖ from the storage unit, and determines whether R(τₘₐₓ) is greater than a second threshold (S203). In a case of YES in any of Step S202 and Step S203, the determination unit 413 determines whether f_{B}(tₖ) is in a predetermined frequency band (S204). As described above, in the present embodiment, the predetermined frequency band means a frequency band equivalent to a period of the breathing motion, for example. In addition, although the first threshold and the second threshold are constants, these constants may be changed as necessary.

In a case of YES in Step S202 or Step S203 and YES in Step S204, an estimation frequency f_{B}(tₖ) output from the determination unit is substituted by the second estimation frequency f_{B}(tₖ) (S205). On the other hand, in a case of NO in Step S202 and Step S203 or NO in Step S204, the estimation frequency f_{B}(tₖ) output from the determination unit is substituted by the first estimation frequency f_{A}(tₖ) (S206).

In the case of NO in Step S202 and Step S203, it is highly possible that the second estimation frequency f_{B}(tₖ) is drastically different from an actual frequency because the second estimation frequency f_{B}(tₖ) differs from the first estimation frequency f_{A}(tₖ) drastically and the correlation analysis is not performed appropriately. In this case, the determination unit 413 adopts the first estimation frequency f_{A}(tₖ). In this way, the frequency estimation unit 41 can estimate a breathing waveform for each period with great accuracy by using the first frequency estimation unit 411 and the second frequency estimation unit 412 in a complementary style so as to estimate a period of a waveform in which fluctuation occurs often such as a breathing motion, the first frequency estimation unit 411 being able to estimate an approximate frequency with low accuracy, the second frequency estimation unit 412 being able to estimate a frequency with great accuracy but being not able to follow irregular change.

The first estimation frequency f_{A}(tₖ) estimated in Step S105 is stored in the storage unit. Subsequently, as shown in the block diagram in FIG. 3, the stored f_{A}(tₖ) is fed back as a frequency of a reference signal to the first frequency estimation unit 411 when a first estimation frequency f_{A}(tₖ₊₁) of a one-dimensional signal r(tₖ₊₁) input at a time of tₖ₊₁ is estimated. In addition, the stored f_{A}(tₖ) is fed back as information for performing correlation analysis to the second frequency estimation unit 412 when the second estimation frequency f_{B}(tₖ₊₁) is estimated.

Note that, a value obtained by integrating a coefficient less than 1 and the first estimation frequency f_{A}(tₖ) may be used as the frequency of the reference signal relating to the feedback. Alternatively, for example, a value obtained by integrating a coefficient less than 1 and the second estimation frequency f_{B}(tₖ) may be used as the frequency of the reference signal. Alternatively, for example, a frequency obtained from a process before or after the first estimation frequency f_{A}(tₖ) or the second estimation frequency f_{B}(tₖ) may be used as the frequency of the reference signal. Alternatively, for example, the frequency to be fed back may be a frequency directly obtained from a beat signal by using another means such as the Fourier transform, or may be a frequency obtained by combining the frequencies by using the addition process, the integration process, and the like.

### (Estimation of Peak Position)

The operation example at the time of estimating the frequency according to the present embodiment has been described. Next, the following describes the operation example at a time of estimating a peak position of the one-dimensional signal r(t) according to the present embodiment on the basis of the estimation frequency f_{E}(tₖ) obtained by the frequency estimation unit 41.

The estimation frequency f_{E}(tₖ) output from the frequency estimation unit 41 is input to the peak position estimation unit 42, subsequently. The peak position estimation unit 42 estimates a peak position on the basis of the estimation frequency f_{E}(tₖ) (S106). Specifically, from the one-dimensional signal r(t), the peak position estimation unit 42 cuts out a criterion signal having a section length equivalent to a period T_{E} corresponding to the estimation frequency f_{E}(tₖ). An end point of the section length is an estimation time tₖ. The peak position estimation unit 42 estimates a peak position of the cut-out criterion signal. More specifically, the peak position estimation unit 42 generates a cosine wave of a predetermined initial phase having the same period T_{E} as that of the criterion signal (in the present embodiment, the initial phase is 0) to calculate a phase difference between the cosine wave and the criterion signal. It is possible to estimate a peak position of the one-dimensional signal r(t) in a criterion signal section, from the time tₖ and the phase difference calculated above.

The peak position estimation unit 42 can store the estimated peak position in the storage unit. The stored peak position is used by the next peak position deciding unit 43.

The peak position estimated by the peak position estimation unit 42 is not always accurate and sometimes includes an error. Accordingly, it is necessary to correct the estimated peak position and decide the peak position.

By using a statistical means, the peak position deciding unit 43 decides a peak position in accordance with distribution of peak positions estimated in the criterion signal section (S107). More specifically, the peak position deciding unit 43 extracts respective peak positions estimated in the past in a range of a section T_{E} of a criterion signal from the storage unit and obtains distribution of the respective peak positions, the section T_{E} having an end point at a time tₖ when a peak position has been estimated. The distribution of the extracted respective peak positions is represented by a histogram divided into unit sections.

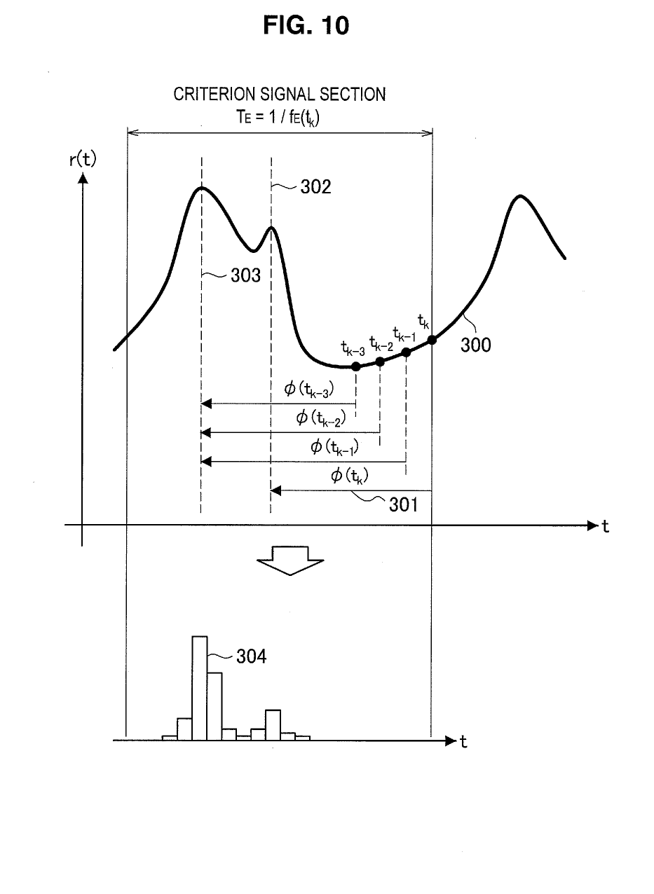

FIG. 10 is an explanatory diagram illustrating an example of a peak position deciding method performed by the peak position deciding unit 43. A curve 300 is the one-dimensional signal r(t). First, the peak position estimation unit 42 assumes that a phase difference φ(tₖ) 301 has been estimated at a time of tₖ. It is assumed that the estimated peak position 302 estimated at this time has been estimated to be a position different from a peak position 303 estimated at another time. If the estimated peak position 302 is not corrected, the estimated peak position 302 is output as it is.

Accordingly, from the storage unit, the peak position deciding unit 43 extracts information on peak positions estimated in the section T_{E} of the criterion signal whose end point is tₖ. Specifically, in a section between tₖ-T_{E} and tₖ, the peak position deciding unit 43 extracts information on peak positions estimated at times tₖ, tₖ₋₁, tₖ₋₂, tₖ₋₃, . . . when the peak positions have been estimated from the storage unit and generates a histogram 304 based on the estimated peak positions that have been extracted. Here, it is assumed that the peak position 303 is a mode in the section of the histogram 304.

Subsequently, the peak position deciding unit 34 calculates an average value of the estimated peak positions, multiplies the histogram 304 by a Gaussian function whose center is the average value and that has given variance, and weights the histogram 304. The mode of the histogram 304 weighted here serves as a decided peak position. The peak position deciding unit 43 can output the most probable peak position even if there is an error in the estimated peak position. The decided peak position that has been obtained is stored in the storage unit.

After the peak position has been decided, the peak interval calculation unit 44 calculates a peak interval on the basis of the decided peak position that has been obtained (S108). Specifically, the peak interval calculation unit 44 calculates a difference between the two decided peak positions that are consecutive to each other and that have been obtained in the peak position deciding unit 43. The decided peak position is called up from the storage unit. The peak interval obtained herein is output as a size of a period of the breathing motion.

After the peak interval has been output, the process returns to Step S101 in a case in which the estimation of period continues (YES in S109), or the use of the apparatus is stopped in a case in which the estimation of period does not continue (NO in S109).

With reference to drawings, the operation example of the period estimation apparatus 20 according to the embodiment of the present invention has been described.

Note that, the operation example according to the embodiment of the present invention has been described on the assumption that the period estimation apparatus 20 estimates a period of an acquired signal in real time. Alternatively, for example, the period estimation apparatus 20 can estimate a period of a past signal stored in the storage unit as batch processing on the basis of information such as the stored signal and the frequencies without performing such real time process.

For example, the period estimation apparatus 20 can store the beat signals D(t) acquired in Step S102 in the storage unit in a chronological order to estimate a period of the beat signal D(t) as the batch processing. As another specific example, the period estimation apparatus 20 can widen the correlation coefficient calculation range 201 to a future after tₖ when estimating the second estimation frequency f_{B}(tₖ) at the time tₖ in Step S105 as the batch processing.

In the above described operation example according to the embodiment of the present invention, the detection target is set to the breathing motion. However, the present invention is not limited thereto. For example, the period estimation apparatus 20 may detect information of other biological motion such as heart beats and body motion. In addition, the period estimation apparatus 20 can detect minute vibrations occurring in various objects.

### <5. Hardware Configuration>

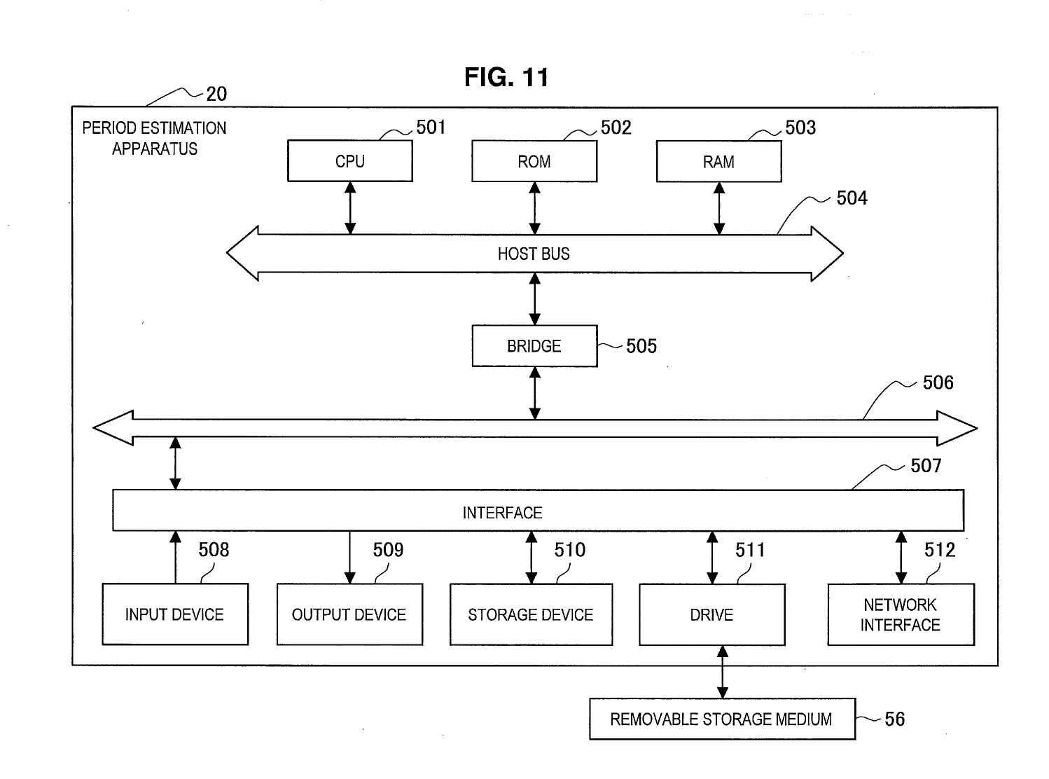

Next, with reference to FIG. 11, a hardware configuration of the period estimation apparatus 20 according to the embodiment of the present invention is described. The information processing in the period estimation apparatus 20 according to the embodiment of the present disclosure is achieved by operating software and the period estimation apparatus 20 cooperatively.

The period estimation apparatus 20 mainly includes a central processing unit (CPU) 501, read only memory (ROM) 502, random access memory (RAM) 503, and a host bus 504. In addition, the period estimation apparatus 20 includes a bridge 505, an external bus 506, an interface 507, an input device 508, an output device 509, a storage device 510, a drive 511, and a network interface 512.

The CPU 501 functions as an arithmetic processing device and a control device to control all of the operating processes in the period estimation apparatus 20 in accordance with various kinds of programs. The CPU 501 may be a microprocessor. The ROM 502 stores programs, operation parameters, and the like used by the CPU 501. The RAM 503 transiently stores programs used when the CPU 501 is executed, and various parameters that change as appropriate when executing such programs. The CPU 501, the ROM 502, and the RAM 503 are connected to each other via the host bus 504 configured of a CPU bus or the like.

The host bus 504 is connected to the external bus 506 such as a Peripheral Component Interconnect/Interface (PCI) bus via the bridge 505. The host bus 504, the bridge 505, and the external bus 506 are not necessarily separated from one another, and their functions may be implemented by one bus.

The input device 508 includes: an input means used by the user for inputting information, such as a mouse, a keyboard, a touch screen, a button, a microphone, a switch, or a lever; an input control circuit configured to generate an input signal based on the user input and to output the signal to the CPU 501; and the like. By operating the input device 508, the user of the period estimation apparatus 20 can input various data into the period estimation apparatus 20 and instruct the period estimation apparatus 20 to perform a processing operation.

The output device 509 includes a display device such as a CRT display device, a liquid crystal display (LCD) device, an OLED device or a lamp. Also, the output device 509 includes an audio output device such as a speaker or a headphone, for example. The output device 509 outputs reproduced contents, for example. Specifically, the display device displays various kinds of information such as reproduced video data by texts or images. On the other hand, the audio output device converts reproduced audio data, text data displayed on the display device or the like into sound and outputs the sound.

The storage device 510 is a device for data storage in the period estimation apparatus 20 according to the present embodiment. The storage device 510 may include a storage medium, a recording device which records data in the storage medium, a reader device which reads data from the storage medium, a deletion device which deletes data recorded in the storage medium, and the like. For example, the storage device is implemented as a hard disc drive (HDD) or a solid state drive (SSD). The storage device 510 stores therein the programs executed by the CPU 501 and various data.

The drive 511 is a reader/writer for the storage medium, and is incorporated in or externally attached to the period estimation apparatus 20. The drive 511 reads information recorded on a removable storage medium 56 that is mounted such as a magnetic disk, an optical disc, a magneto-optical disk, or semiconductor memory, and outputs the information to the RAM 503. The drive 511 also writes information to the removable storage medium 56.

The network interface 512 is, for example, a communication interface including a communication device and the like for connection to another apparatus. Further, the network interface 512 may be a communication device that supports a wireless local area network (LAN), a communication device that supports long term evolution (LTE), a communication device that supports near field communication, or a wire communication device that performs wired communication.

In the embodiment of the present invention, the period estimation apparatus 20 is achieved by the CPU 501, the ROM 502, the RAM 503, the storage device 510, and the like, for example.

The example of the hardware configuration capable of achieving the functions of the period estimation apparatus 20 according to the embodiment of the present invention has been described. Each of the structural elements described above may be configured by using a general purpose component or may be configured by hardware specialized for the function of each of the structural elements. Thus, it is possible to appropriately modify a hardware configuration to be used according to technical levels whenever the present embodiment is implemented.

### <5. Conclusion>

When a frequency is estimated by comparing with a reference signal, an accurate frequency is not specified since a breathing signal is represented approximately. However, it is expected to estimate a frequency and phase that are not far off accurate one since a sketch of a waveform can be maintained. Alternatively, when a period is estimated from correlation, it is difficult to embrace phase concept under a situation in which the period changes, and an erroneous detection of a multiple of a period or an erroneous detection of a frequency due to the apparatus fitted with a fine structure may occur. However, it is expected that there is a peak in a correlation coefficient in a correct period in a case in which a waveform in the periods is sufficiently stable. Therefore, according to the embodiment of the present invention in which the both estimates are combined, it is possible to accurately estimate a period with low erroneous detection by adopting the first estimation frequency and the second estimation frequency in a complementary style, the first estimation frequency being estimated on the basis of a phase difference between the one-dimensional signal and the reference signal and the temporal change in the phase difference, the second estimation frequency being calculated by performing correlation analysis on the one-dimensional signal and the comparative signal having the section length equivalent to a period corresponding to the first estimation frequency. Accordingly, it is possible to accurately estimate a period of a waveform in which fluctuation occurs often such as the breathing motion, for each period.

Heretofore, preferred embodiments of the present invention have been described in detail with reference to the appended drawings, but the present invention is not limited thereto. It should be understood by those skilled in the art that various changes and alterations may be made without departing from the spirit and scope of the appended claims.

In addition, it is also possible to create a computer program for causing hardware such as the CPU 501, ROM 502, and RAM 503, which are embedded in the period estimation apparatus 20, to execute functions equivalent to the structural elements of the period estimation apparatus 20. Moreover, it may be possible to provide a storage medium (non-transitory media) having the computer program stored therein.

Note that, steps listed in the a flow diagrams and flow charts in the present specification not only include those processes that are performed chronologically in the order they are listed, but also include those processes that may not be performed chronologically but are performed in parallel or individually. It is understood that even steps that are processed chronologically can be in some cases performed in a different order as necessary.

What is claimed is:

1. A period estimation apparatus comprising:
a first frequency estimation unit that estimates a first frequency of a one-dimensional signal on the basis of a phase difference between the one-dimensional signal and a reference signal and a temporal change in the phase difference; and
a second frequency estimation unit that estimates a second frequency of the one-dimensional signal by cutting out a comparative signal having a section length equivalent to a first period corresponding to the first frequency from the one-dimensional signal, and calculating a correlation coefficient between the comparative signal and the one-dimensional signal having the section length.

2. The period estimation apparatus according to claim 1,
wherein the first frequency estimation unit generates the reference signal having a frequency depending on the estimated first frequency.

3. The period estimation apparatus according to claim 1,
wherein, in a case in which the estimated first frequency is not present in a predetermined frequency band, the first frequency estimation unit estimates, as the first frequency of the one-dimensional signal, a frequency of the reference signal used by the first frequency estimation unit.

4. The period estimation apparatus according to claim 1,
wherein the second frequency estimation unit estimates a second frequency of the one-dimensional signal on the basis of a time lag at a time when the correlation coefficient becomes maximum in a range determined by the first period.

5. The period estimation apparatus according to claim 1, further comprising:
a determination unit that determines any of the first frequency and the second frequency as an estimation frequency of the one-dimensional signal on the basis of the first frequency, the second frequency, and the correlation coefficient at a time when the second frequency has been estimated.

6. The period estimation apparatus according to claim 1, further comprising:
a Doppler sensor that radiates a radiation wave toward an object, and outputs a beat signal having a frequency of a difference between a frequency of the radiation wave and a frequency of a reflection wave reflected by the object that has received the radiation wave;
a beat signal acquisition unit that acquires the beat signal output from the Doppler sensor; and
a signal conversion unit that converts the acquired beat signal into the one-dimensional signal.

7. The period estimation apparatus according to claim 6, further comprising:
a filter unit that reduces a low-frequency component in the beat signal and outputs a corrected signal obtained by reducing the low-frequency component.

8. The period estimation apparatus according to claim 7,
wherein the signal conversion unit
performs principal component analysis on the corrected signal represented as a two-dimensional vector, and
converts the corrected signal into the one-dimensional signal having an output value that is a value of a principal component direction of the corrected signal converted by an eigenvector obtained from the principal component analysis.

9. The period estimation apparatus according to claim 7,
wherein the signal conversion unit converts the corrected signal into the one-dimensional signal having an output value that is a solution of a continuous function of a product of strength of the corrected signal and temporal differentiation of an argument of the corrected signal on a two-dimensional surface.

10. The period estimation apparatus according to claim 6,
wherein motion of the object is breathing motion of a living body.

11. The period estimation apparatus according to claim 5, further comprising:
a peak position estimation unit that cuts out a criterion signal having a section length equivalent to a period corresponding to the estimation frequency from the one-dimensional signal, and estimates a peak position of the one-dimensional signal on the basis of a phase difference between the criterion signal and a cosine wave having the estimation frequency and a predetermined initial phase.

12. The period estimation apparatus according to claim 11, further comprising:
a peak position deciding unit that extracts respective peak positions estimated in the past in a section of the criterion signal used at a time when the peak position estimation unit has estimated the peak position, and decides the peak position on the basis of distribution of the respective peak positions that have been extracted.

13. The period estimation apparatus according to claim 12, further comprising:
a peak interval calculation unit that calculates, as an peak interval, a difference between the two decided peak positions that are consecutive to each other.

14. A period estimation method comprising:
estimating a first frequency of a one-dimensional signal on the basis of a phase difference between the one-dimensional signal and a reference signal and a temporal change in the phase difference; and
estimating a second frequency of the one-dimensional signal by cutting out a comparative signal having a section length equivalent to a first period corresponding to the first frequency from the one-dimensional signal, and calculating a correlation coefficient between the comparative signal and the one-dimensional signal having the section length.

15. A storage medium having a program stored therein, the program causing a computer to function as:
a first frequency estimation unit that estimates a first frequency of a one-dimensional signal on the basis of a phase difference between the one-dimensional signal and a reference signal and a temporal change in the phase difference; and
a second frequency estimation unit that estimates a second frequency of the one-dimensional signal by cutting out a comparative signal having a section length equivalent to a first period corresponding to the first frequency from the one-dimensional signal, and calculating a correlation coefficient between the comparative signal and the one-dimensional signal having the section length.
